(19) United States
(12) Patent Application Publication
(10) Pub. No.: US 2026/0325744 A1
(43) Pub. Date: Oct. 22, 2026

(54) Method to Provide for Drug Product Filling Into Body-Worn Drug Delivery Device

(71) Applicant: Becton, Dickinson and Company, Franklin Lakes, NJ (US)

(72) Inventors: Stuart Plascott, Foxrock (IE); Christian Sandmann, Wayne, NJ (US)

(21) Appl. No.: 19/110,556

(22) PCT Filed: Sep. 22, 2023
(86) PCT No.: PCT/US2023/033445

§ 371 Date: Mar. 11, 2025

Related U.S. Application Data
(60) Provisional application No. 63/409,369, filed on Sep. 23, 2022.

(51) Int. Cl. A61M 5/142

(52) U.S. Cl. CPC A61M 5/14248; A61M 2209/045

(57) ABSTRACT

A system for filling an on-body injector includes the on-body injector having a medicament reservoir, a fill cartridge having an internal chamber containing a medicament, and a filling station for receiving the on-body injector and the fill cartridge. The filling station includes an injector socket for receiving the on-body injector, a cartridge socket for receiving the fill cartridge, a fluid path section providing fluid communication between the injector socket and the cartridge socket and a pump operatively engaging the fluid path section to transfer the medicament from the internal chamber of the fill cartridge to the medicament reservoir of the on-body injector.

### CROSS-REFERENCE TO RELATED APPLICATION

The present application claims priority to U.S. Provisional Application Ser. No. 63/409,369 entitled “A Method to Provide for Drug Product Filling Into Body-Work Drug Delivery Device” filed Sep. 23, 2022, the entire disclosure of which is hereby incorporated by reference in its entirety.

### BACKGROUND OF THE INVENTION

### Field of the Invention

The present disclosure relates to a filling station for drug delivery devices and to a method for filling drug delivery devices.

### Description of Related Art

Wearable medical injection devices, which may be variously referred to as on-body injectors, wearable injectors, path pumps, etc., are commonly used in treatment of diseases such as diabetes where continuous infusion of a medicament (e.g. insulin) is indicated.

In the case of diabetes, on-body insulin pumps are typically designed to administer insulin at a basal rate and in a bolus dose. During basal administration, insulin is delivered continuously over a predetermined time period (e.g. 24 hours) to maintain the diabetic patient's blood glucose levels in a consistent range. Basal administration can result in less variation within the blood glucose range compared to a single injection, and eliminates the need for multiple needle sticks over the predetermined time period. During bolus administration, insulin is delivered in a discrete injection dosage. A bolus dose may be indicated, for example, when a diabetic patient consumes a meal to balance the consumed carbohydrates.

Patch pumps are designed to be a self-contained unit that is worn by the patient, and are preferably small as to not interfere with the activities of the user. As such, the medicament capacity of patch pumps is limited, and patch pumps must be filled/refilled by health care providers once all of the medicament has been administered.

It would be desirable if patch pumps could be filled and/or refilled by the patient or the patient's caregivers without the need for intervention of conventional healthcare providers.

### SUMMARY

Embodiments of the present disclosure are directed to a system for filling an on-body injector. The system includes the on-body injector having a medicament reservoir, a fill cartridge having an internal chamber containing a medicament and a filling station for receiving the on-body injector and the fill cartridge. The filling station includes an injector socket for receiving the on-body injector, a cartridge socket for receiving the fill cartridge, a fluid path section providing fluid communication between the injector socket and the cartridge socket, and a pump operatively engaging the fluid path section to transfer the medicament from the internal chamber of the fill cartridge to the medicament reservoir of the on-body injector.

In some embodiments, the pump includes one or more rollers configured to engage a resilient tubing section of the fluid path section.

In some embodiments, the injector socket includes a needle for piercing a septum or port of the on-body injector.

In some embodiments, the cartridge socket includes a needle for piercing a septum or stopper of the fill cartridge.

In some embodiments, the injector socket, the cartridge socket, and the fluid path section are provided on a removable transfer tray.

In some embodiments, the filling station includes a base for receiving the transfer tray, and a lid pivotally connected to the base.

In some embodiments, the filling station includes an inflation chamber configured to deliver a pre-fill inflation volume of air to the on-body injector.

In some embodiments, the pre-fill inflation volume of air is approximately 100 μL.

In some embodiments, the medicament reservoir of the on-body injector has a volume of approximately 3 mL.

In some embodiments, the filling station includes a controller for actuating the pump.

In some embodiments, the controller is configured to actuate the pump to fill the medicament reservoir of the on-body injector to approximately 10 psi.

In some embodiments, the controller is configured to run the pump in reverse to evacuate air from the fluid path section.

In some embodiments, the filling station further comprises a reverse check valve through which the air is evacuated.

In some embodiments, the filling station further includes an auxiliary socket configured for receiving an auxiliary cartridge and at least one valve movable between a first position and a second position. In the first position of the at least one valve, the cartridge socket is in fluid communication with the auxiliary socket, and the injector socket is isolated. In the second position of the valve, the cartridge socket is in fluid communication with the injector socket.

Other embodiments of the present disclosure are directed to a filling station for filling an on-body injector. The filling station includes a removable transfer tray includes an injector socket for receiving the on-body injector, a cartridge socket for receiving a fill cartridge, a fluid path section providing fluid communication between the injector socket and the cartridge socket, and a pump operatively engaging the fluid path section to transfer the medicament from an internal chamber of the fill cartridge to a medicament reservoir of the on-body injector.

In some embodiments, the pump includes one or more rollers configured to engage a resilient tubing section of the fluid path section.

In some embodiments, the injector socket includes a needle for piercing a septum or port of the on-body injector.

In some embodiments, the cartridge socket includes a needle for piercing a septum or stopper of the fill cartridge.

In some embodiments, the filling station further includes a base for receiving the transfer tray, and a lid pivotally connected to the base.

In some embodiments, the filling station further includes an inflation chamber configured to deliver a pre-fill inflation volume of air to the on-body injector.

In some embodiments, the pre-fill inflation volume of air is approximately 100 μL.

In some embodiments, the filling station includes a controller for actuating the pump. The controller is configured to actuate the pump to fill the medicament reservoir of the on-body injector to approximately 10 psi.

In some embodiments, the controller is configured to run the pump in reverse to evacuate air from the fluid path section.

Further details and advantages of the various examples described in detail herein will become clear upon reviewing the following detailed description of the various examples in conjunction with the accompanying drawing figures.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a filling station according to an embodiment of the present disclosure;

FIG. 2 is a schematic diagram of a transfer tray of the filling station of FIG. 1, loaded with an on-body injector and a fill cartridge;

FIG. 3 is a fluid path diagram of the transfer tray of FIG. 2, according to an embodiment of the present disclosure;

FIG. 4 is a perspective exploded view of the filling station of FIG. 1 in an open position, with portions of the base and lid removed for clarity;

FIG. 5 is a perspective exploded view of the filling station of FIG. 1 in an open position, with portions of the base and lid removed for clarity;

FIG. 6 is a perspective view of the filling station of FIG. 1 in an open position, with portions of the base and lid removed for clarity;

FIG. 7 is a perspective view of the filling station of FIG. 1 in a closed position, with portions of the base and lid removed for clarity;

FIG. 8 is a perspective view of the filling station of FIG. 1 in an open position, with portions of the base and lid removed for clarity;

FIG. 9 is a perspective view of the filling station of FIG. 1 in an open position, with portions of the base and lid removed for clarity;

FIG. 10 is a perspective exploded view of a filling station according to an embodiment of the present disclosure;

FIG. 11 is a perspective view of the transfer tray of FIG. 10;

FIG. 12 is a perspective view of the transfer tray of FIG. 10, in an open position;

FIG. 13 is a perspective view of the transfer tray of FIG. 10, in a closed position;

FIG. 14 is an exploded perspective view of the transfer tray of FIG. 10;

FIG. 15 is a perspective and side view of a filling station according to an embodiment of the present disclosure;

FIG. 16 is a fluid path diagram of the filling station of FIG. 15;

FIG. 17 is a perspective exploded view of a filling station according to an embodiment of the present disclosure;

FIG. 18 is a fluid path diagram of the filling station of FIG. 17;

FIG. 19 is a perspective exploded view of a filling station according to an embodiment of the present disclosure;

FIG. 20 is a fluid path diagram of the filling station of FIG. 17;

FIG. 21 is a fluid path diagram of the transfer tray of FIG. 2, according to an embodiment of the present disclosure, with a valve thereof in a first position; and

FIG. 22 is the fluid path diagram of FIG. 21, with the valve in a second position.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

All numbers used in the specification and claims are to be understood as being modified in all instances by the term “about”. The terms “approximately”, “about”, and “substantially” mean a range of plus or minus ten percent of the stated value. Further, the term “substantially equal” and like terms mean that the compared values or dimensions are within a range of plus or minus ten percent of one another.

The terms “first”, “second”, and the like are not intended to refer to any particular order or chronology, but refer to different conditions, properties, or elements.

As used herein with reference to an injection apparatus such as a syringe, the term “proximal” refers to an end of the apparatus farthest from the outlet, or to a direction toward the end of the apparatus farthest from the outlet. As used herein with reference to an injection apparatus such as a syringe, the term “distal” refers to an end of the device or apparatus closest to the outlet, or to a direction toward the end of the apparatus closest to the outlet.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

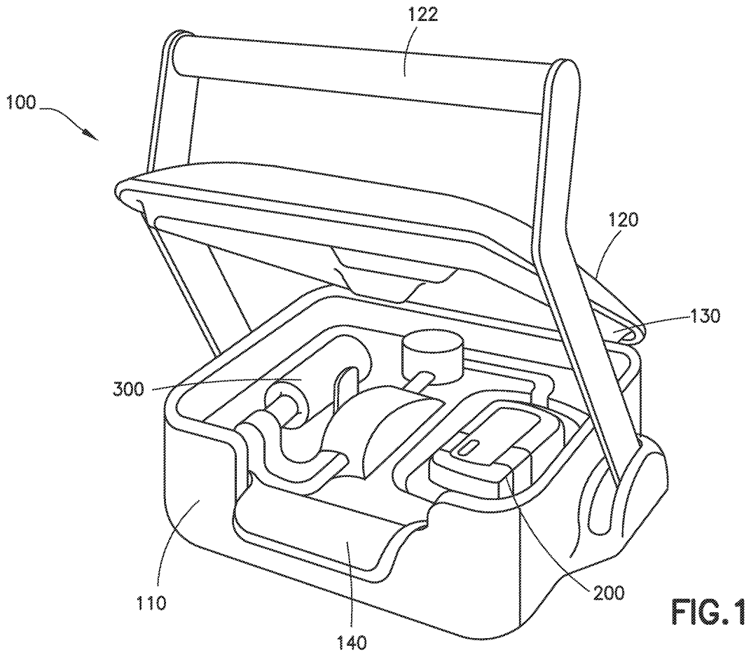

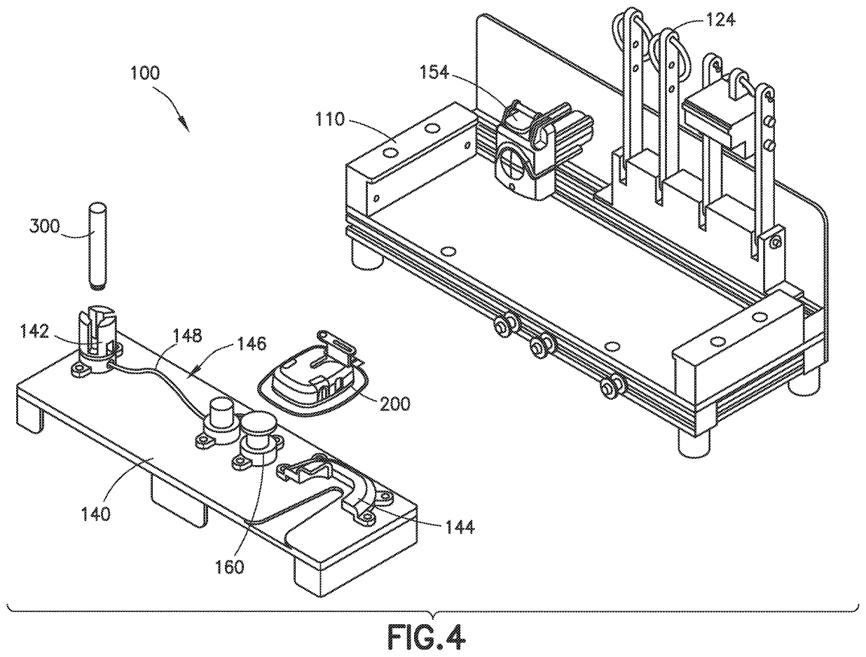

Referring first to FIG. 1, an embodiment of an injector filling station 100 is illustrated in accordance with an embodiment of the present disclosure. The filling station 100 can include a base 110 and a lid 120. The lid 120 can be pivotally connected to the base 110, for example by a rotational hinge 130. In some embodiment, the lid 120 includes a handle 122 to assist a user in manipulating the lid 120. The base 110 and/or the lid 120 can removably support a transfer tray 140. The tray 140 includes various features for supporting and removably retaining an injector, more particularly an on-body injector 200 (which may also be referred to hereinafter as a patch pump). The tray 140 further includes various features for supporting and removably retaining a fill cartridge 300 (containing, for example, a medicament), and for establishing fluid communication between the fill cartridge 300 and a medicament reservoir of the on-body injector 200. In use, a user (typically a patient or the patient's caregiver) loads an empty on-body injector 200 and the fill cartridge 300 onto the tray 140. The user then inserts the tray 140 into the base 110 and/or the lid 120 of the filling station 100. The tray 140, the base 110, and/or the lid 120 include various structures and components for transferring the medicament from the fill cartridge 300 into the reservoir of the on-body injector 200. In some embodiments, transfer of the medicament from the fill cartridge 300 into the medicament reservoir of the on-body injector 200 may occur automatically upon closure of the lid 120, as will be discussed in greater detail herein with reference to FIGS. 4-9.

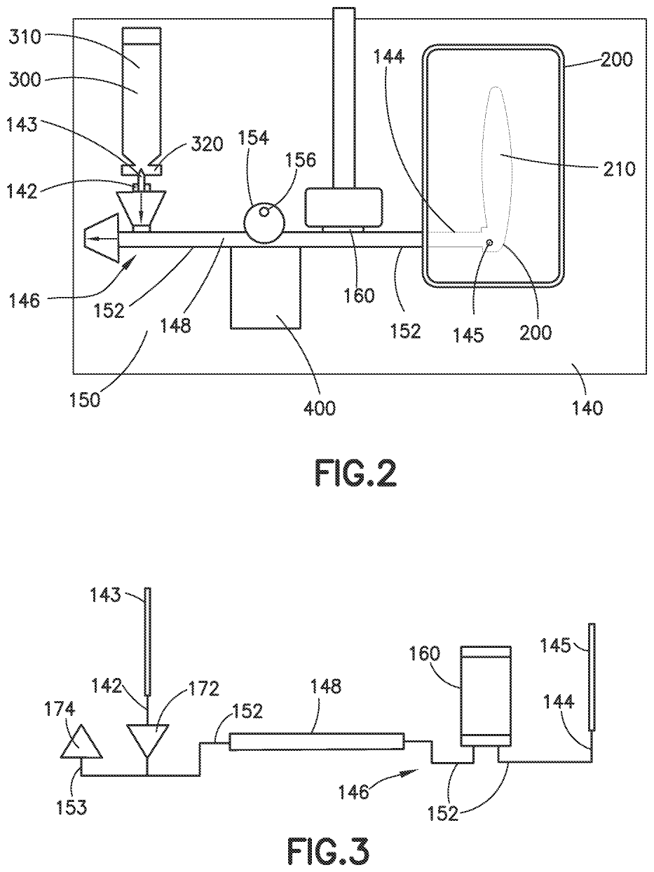

Referring now to FIGS. 2 and 3, the on-body injector 200 and the fill cartridge 300 can be removably connected to the tray 140 such that a fluid path is established between an internal chamber 310 of the fill cartridge 300 and a medicament reservoir 210 of the on-body injector 200. The tray 140 includes a cartridge socket 142 for removably receiving the fill cartridge 300 and an injector socket 144 for removably receiving the on-body injector 200. The tray 140 further includes a fluid path section 146 providing fluid communication between the cartridge socket 142 and the injector socket 144. As shown in FIG. 3, the cartridge socket 142 can include a needle 143 for piercing a septum or stopper 320 of the fill cartridge 300 to provide fluid communication between the fluid path section 146 and the interior chamber 310 of the fill cartridge 300. Similarly, the injector socket 144 can include a needle 145 for piercing a septum or port 220 of the on-body injector 200 to provide fluid communication between the fluid path section 146 and the medicament reservoir 210 of the on-body injector 200. The fluid path section 146 can include one or more fluid channels 152 molded and/or formed into a base material 150 of the tray 140, and/or one or more tubing sections 148 connected to the base material 150.

With continued reference to FIG. 2, the tray 140 and/or other components of the filling station 100 include a pump 154 or other component for transferring medicament out of the fill cartridge 300 and into the on-body injector 200. The pump 154 can be disposed in the fluid path section 146. The pump 154 is controlled by an electronic controller 400 programmed or configured to actuate the pump 154 until a fill or refill operation has been completed, and/or to abort the fill or refill operation in the event of an error in loading or filling the on-body injector 200. In the embodiment shown in FIG. 2, the pump 154 is a peristaltic pump having one or more rollers 156 configured to engage a resilient tubing section 148 of the fluid path section 146 in order to draw medicament out of the fill cartridge 300 and inject the medicament into the on-body injector 200.

With continued reference to FIG. 2, the tray 140 can further include an inflation chamber 160 configured to inject a pre-inflation volume of air into on-body injector 200 prior to filling the on-body injector 200 with medicament. The pre-inflation volume may be used to generate a controlled volume of air in the reservoir of the on-body injector 200. The pre-inflation volume may additionally or alternatively be used to occupy dead space on the reservoir of the on-body injector 200 to assist in ejecting more medicament from the reservoir during an injection procedure, especially towards an end of the injection procedure when the reservoir is nearly empty. In embodiments in which the on-body injector 200 has a reservoir volume of approximately 3 milliliters (mL), the inflation chamber 160 may be configured to inject a pre-fill inflation volume of approximately 100 microliters (μL) of air per injector fill operation. The inflation chamber 160 is disposed in fluid communication with the fluid path section 146 between the cartridge socket 142 and the injector socket 144 such that the inflation chamber 160 supplies air to the on-body injector 200 via the fluid path section 146. In the illustrated embodiment, the inflation chamber includes a piston 162 that moves axially to eject pressurized air from the inflation chamber 160.

With continued reference to FIG. 2, the tray 140 can further include a forward check valve 172 disposed in the fluid path section 146 between the on-body injector 200 and the fill cartridge 300. The forward check valve 172 prevents flow of air and/or medicament back from the on-body injector 200 into the fill cartridge 300. The tray 140 can further include a reverse check valve 174 disposed in the fluid path section 146 to allow air to escape from the on-body injector 200 via the fluid path section 146 via the pump 154. The reverse check valve 174 can be disposed in a branch channel 153 of the fluid path section 146 arranged such that fluid can flow from the on-body injector 200 to the reverse check valve 174 without flowing into the fill cartridge 300, and such that fluid can flow from the fill cartridge 300 to the reverse check valve 174 without flowing into the on-body injector 200. In other words, the on-body injector 200 and the fill cartridge 300 are in parallel fluid circuits relative to the reverse check valve 174.

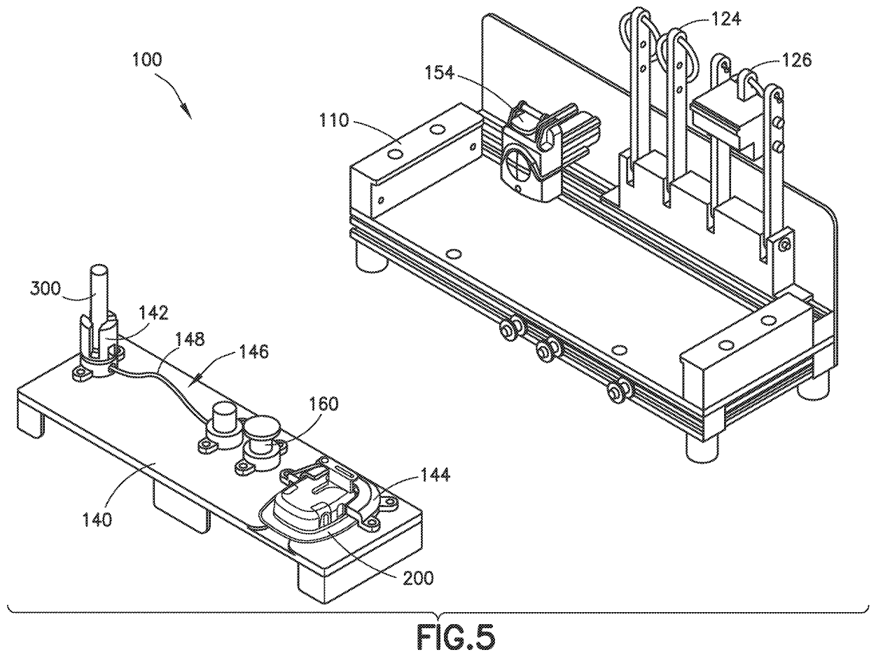

Referring now to FIGS. 4-9, loading of the filling station 100 is illustrated in accordance with an embodiment of the present disclosure. Referring first to FIG. 4, the tray 140 can be removed from (or supplied separately from) the base 110 and the lid 120 to allow access to the cartridge socket 142 and the injector socket 144. As shown in FIGS. 4 and 5, the cartridge socket 142 can include a port with which the fill cartridge 300 has a snap-fit connection. In other embodiments, the cartridge socket 142 can include a bayonet connector, and threaded fitting, or other secure mechanical connection to engage and hold the fill cartridge 300. The cartridge socket 142 may exhibit tactile feedback, such as a snap fit, to confirm to the user that the fill cartridge 300 is properly inserted into the cartridge socket 142. Connection of the fill cartridge 300 to the cartridge socket 142 can automatically cause the needle 143 of the cartridge socket 142 to pierce the septum or stopper of the fill cartridge 300. Likewise, the injector socket 144 can include a port with which the on-body injector 200 has a snap-fit connection. The cartridge socket 142 may exhibit tactile feedback, such as a snap fit, to confirm to the user that the on-body injector 200 is properly inserted into the injector socket 144. Connection of the on-body injector 200 to the injector socket 144 can automatically cause the needle 145 of the injector socket 144 to pierce the septum of the on-body injector 200.

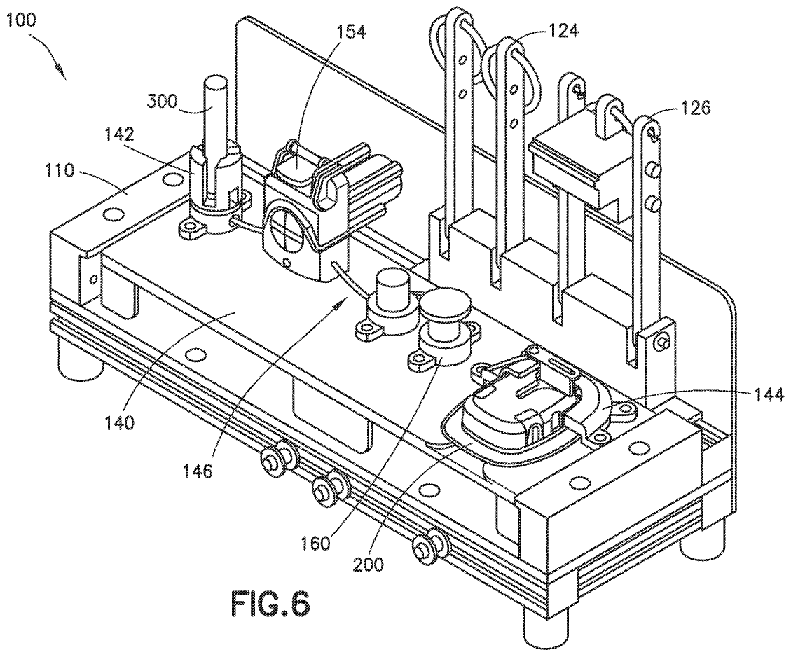

Referring now to FIG. 6, once the on-body injector 200 and the fill cartridge 300 are connected to the injector socket 144 and the cartridge socket 142, respectively, the tray 140 can be inserted into the base 110 of the filling station 100. The base 110 may include locating features such as detents and/or projections to ensure that the tray 140 is properly aligned with the operative components of the base 110 and/or the lid 120. For example, the tray 140 must be positioned within the base 110 such that the resilient tubing section 148 of the fluid path section 146 operatively aligns and engages with the pump 154. Similarly, the tray 140 must be positioned within the base 110 such that the inflation chamber 160 is operatively aligned with a corresponding actuating lever 124 of the lid 120, and such that the on-body injector 200 is operatively aligned with a corresponding retaining lever 126 of the lid 120. In some embodiments, the base 110, the lid 120, and/or the tray 140 can exhibit tactile feedback, such as a snap-fit, to confirm to the user that the tray 140 is properly inserted. In some embodiments, the base 110, the lid 120, and/or the tray 140 can include electromechanical devices, e.g. limit switches, configured to prevent performance of a fill or refill operation if the tray 140 is not properly positioned with the base 110.

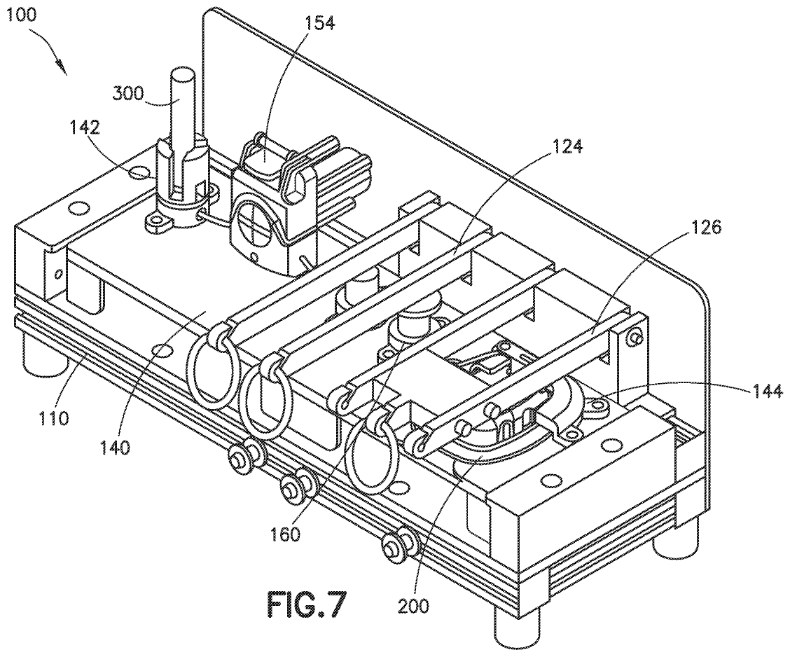

Referring now to FIG. 7, once the tray 140 is properly positioned within the base 110, the lid 120 can be closed by the user to initiate a fill or refill operation. Closure of the lid 120 causes the retaining lever 126, which may be integrally formed with the lid 120, to engage the on-body injector 200 to prevent movement of the on-body injector 200 during the fill or refill operation. Closure of the lid 120 also causes the actuating lever 124, which may be integrally formed with the lid 120, to engage the inflation chamber 160 and thereby inject the pre-fill inflation volume of air into the medicament reservoir 210 of the on-body injector 200. The retaining lever 126 and the actuating lever 124 can be timed such that, during closure of the lid 120, the retaining lever 126 engages the on-body injector 200 prior to the actuating lever 124 engaging the inflation chamber 160 so that the on-body injector 200 is fully supported by the retaining lever 126 prior to the pre-fill inflation volume of air being delivered.

Once the lid 120 is closed, as shown in FIG. 7, the fill or refill operation can commence. The filling station 100 may include one or more sensors (not shown) configured to detect closure of the lid 120 and initiate the controller 400 to perform the fill or refill operation. During the fill or refill operation, pre-fill inflation volume of air is delivered to the on-body injector 200 as described in connection with FIG. 7. The controller 400 can initially actuate the pump 154 in a reverse direction for a predetermined time to evacuate air from the on-body injector 200 and the fluid path section 146 of the tray 140. After the predetermined time, the controller can actuate the pump 154 in a forward direction to draw medicament from the internal chamber 310 of the fill cartridge 300 and inject the medicament into the medicament reservoir 210 of the on-body injector 200. The controller 400 can actuate the pump 154 until the medicament reservoir 210 is filled to a predetermined pressure, for example approximate 10 psi. Filling of the on-body injector 200 is then complete, and the controller stops actuation of the pump 154. The pump 154 can be controlled throughout the fill or refill operation by the controller. In some embodiments, the controller may be configured to emit sensory feedback, for example illuminating a light or emitting a sound, to indicate to the user that the on-body injector 200 has been filled.

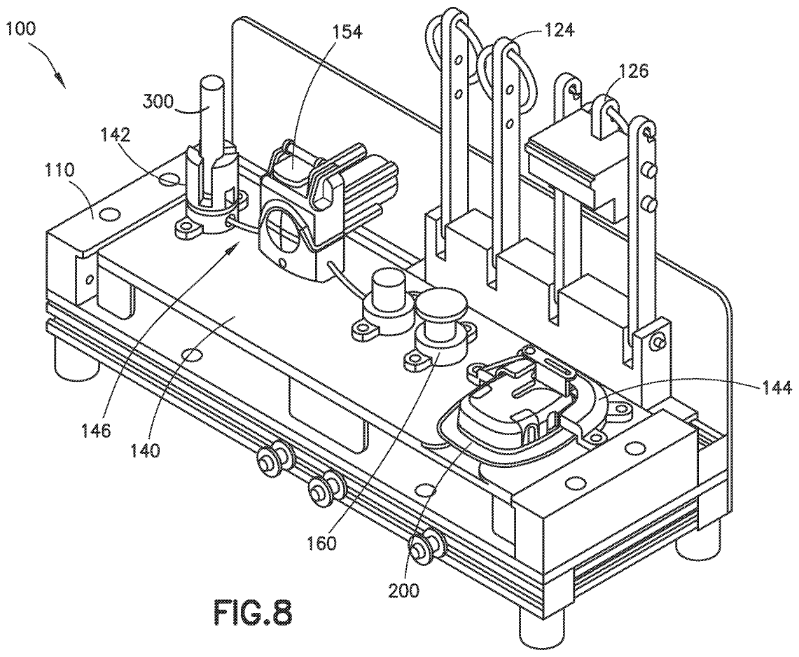

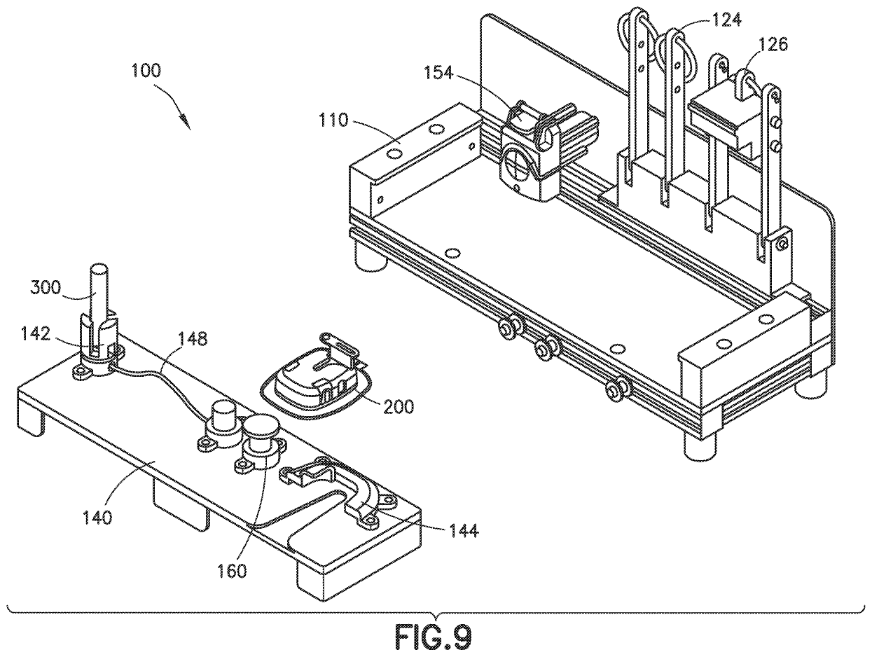

Referring now to FIG. 8, once fill of the on-body injector 200 is complete, the lid 120 may be opened either automatically by the controller or manually by the user. Opening of the lid 120 releases the retaining lever 126 from the on-body injector 200 and releases the actuation lever 124 from the inflation chamber 160. If the lid 120 is opened by the user prior to the fill or refill operation being completed, the controller may also halt operation of the pump 154 as a safety measure to stop further filling of the on-body injector 200. With the retaining lever 126 and the actuation lever 124 disengaged from the on-body injector 200 and the inflation chamber 160, respectively, the tray 140 can be removed from the base 110 as shown in FIG. 9. The on-body injector 200 and the fill cartridge 300 can then be detached from the tray 140, and the on-body injector 20 can be attached to the patient and used as if the on-body injector 200 had been filled by a health care provider. In some embodiments, the fill cartridge 300 can contain sufficient medicament to fill multiple on-body injectors 200, in which case the fill cartridge 300 can be detached from the tray 140 and stored for future fill or refill operations. In some embodiments, the fill cartridge 300 contains only enough medicament to perform a single fill or refill operation, so the fill cartridge 300 can be disposed of after the fill or refill operation. In some embodiments, the tray 140 may also be disposable and may be replaced after each fill or refill operation or after a predetermined number of fill or refill operations.

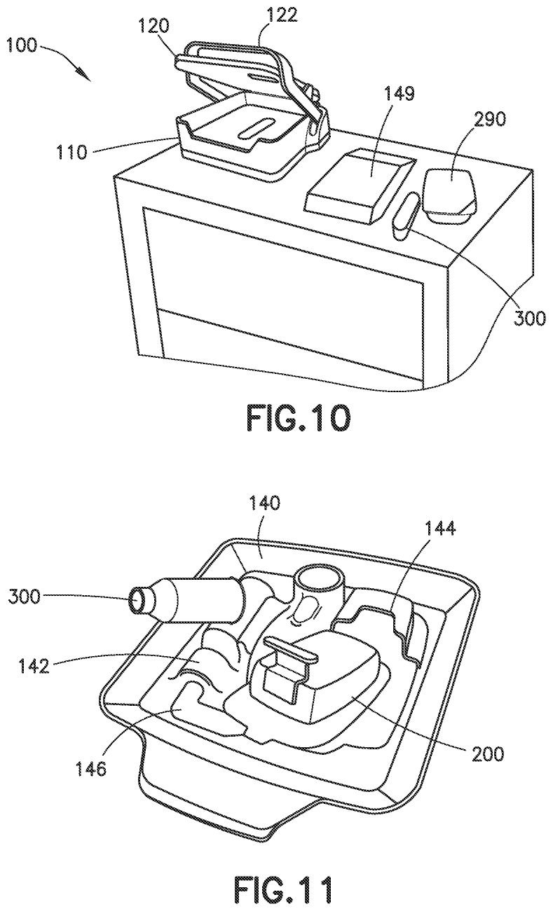

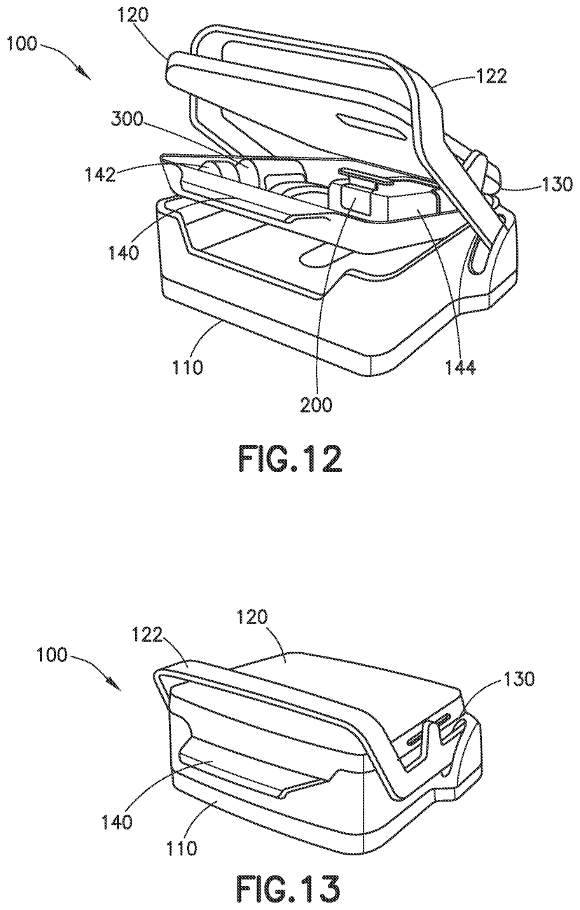

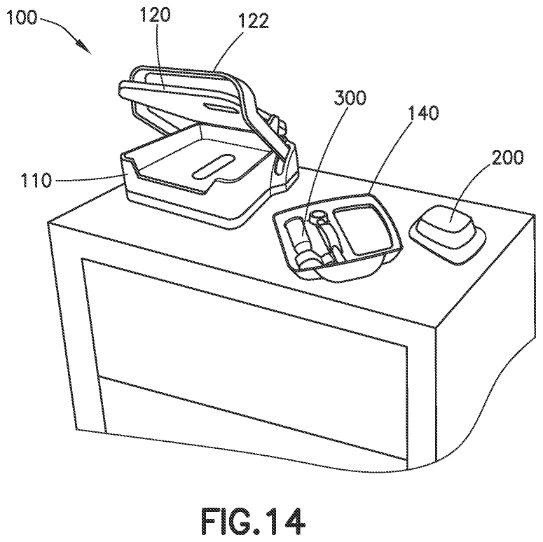

Referring now to FIGS. 10-14, a method or process of filling or refilling the on-body injector 200 is illustrated from the perspective of the user (e.g. the patient or the patient's caregiver) operating the filling station 100. As shown in FIG. 10, the user first obtains the tray 140, the on-body injector 200, and the fill cartridge 300. One or more of the tray 140, the on-body injector 200, and the fill cartridge 300 may be disposable components supplied in individual packaging. For example, the tray 140 may be supplied in tray packaging 149, and the on-body injector 200 may be supplied in injector packaging 290. The user can remove the tray 140, the on-body injector 200, and the fill cartridge 300 from any associated packaging, and then, as shown in FIG. 11, the user can load the on-body injector 200 and the fill cartridge 300 onto the tray 140 in the manner described herein in connection with FIGS. 4 and 5. As shown in FIG. 12, the user can open the lid 120 of the filling station 100 and insert the tray 140 loaded with the on-body injector 200 and the fill cartridge 300 into the base 120. As shown in FIG. 13, the user can close the lid 120 and initiate performance of the fill or refill operation, as described herein in connection with FIGS. 6-8. As shown in FIG. 14, after the fill or refill operation has been performed, the user can open the lid 120 (or, in some embodiments, the lid 120 may be automatically opened by the controller) and the user can remove the tray 140 loaded with the on-body injector 200 and the fill cartridge 300. The user can then remove the on-body injector 200 from the tray 140, and proceed to attach the on-body injector 200 to the patient to administer the medicament from the on-body injector 200. The user can also dispose of the tray 140 and/or the fill cartridge 300 if those components have reached their end-of-life. As described herein, the tray 140 and/or the fill cartridge 300 may be disposable components configured for use in a predetermined number (e.g., one) of fill operations, after which the tray 140 and/or the fill cartridge 300 should be discarded for hygienic concerns.

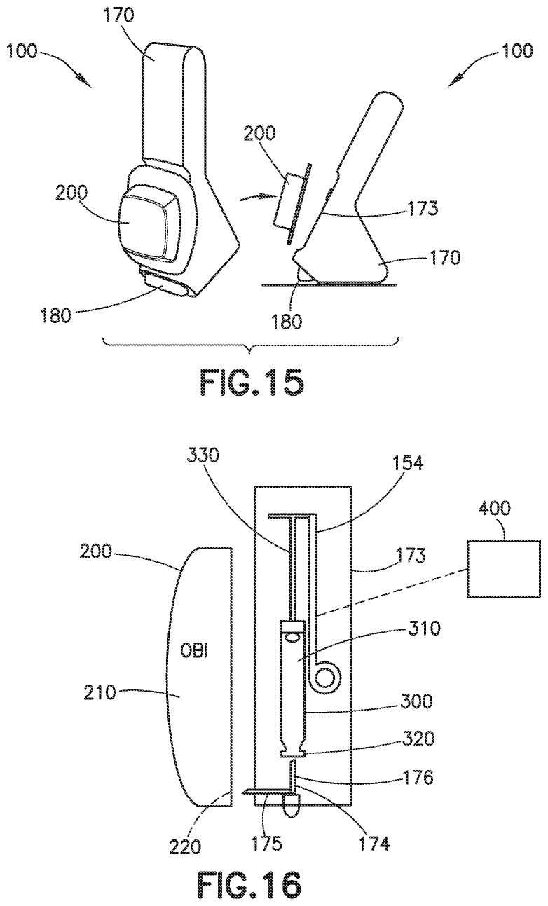

Referring now to FIGS. 15-20, alternative embodiments of the filling station 100 are illustrated. The embodiments of the filling station 100 shown in FIGS. 15-20 can operate on the same principles and with the same purpose as the embodiments described in connection with FIGS. 1-14. Namely, the embodiments of FIGS. 15-20 are intended for filling the on-body injector 200. Components of the embodiments of FIGS. 15-20 may correspond to parts having the same reference numerals in FIGS. 1-14. Referring first to the embodiment shown in FIGS. 15 and 16, an embodiment of the filling station 100 includes a base 170 having a receiving surface 172 for receiving and supporting the on-body injector 200. The base 170 has an integrated or removable fluid path section 174, analogous in function to the fluid path section 146 of the embodiment described herein in connection with FIGS. 2 and 3.

With continued reference to FIGS. 15 and 16, the fluid path section 174 provides fluid communication between a needle 175 piercing the septum or port 220 of the on-body injector 200, and the cartridge socket 176. The cartridge socket 176 is configured to support the fill cartridge 300 and provide fluid communication between the fluid path section 174 and the internal chamber 310 of the fill cartridge 300. As shown in FIG. 16, the cartridge socket 176 can include a needle 177 for piercing the septum or stopper 320 of the fill cartridge 300 to access the internal chamber 310. The pump 154 in the embodiment of FIGS. 15 and 16 may include a ram for driving a plunger 330 through the internal chamber 310 of the fill cartridge 300 to inject the medicament contained in the internal chamber 310 of the fill cartridge 300 into the medicament reservoir 210 of the on-body injector 200 via the fluid path section 174.

With continued reference to FIGS. 15 and 16, the base 170 can include a button or switch 180 that can be actuated by the user to initiate the fill or refill operation once the on-body injector 200 has been connected to the base 170. Actuation of the button or switch 180 can cause the controller 400 to actuate the pump 154 to inject the medicament from the fill or refill cartridge 300 into the on-body injector 200. Once the fill or refill operation is complete, the user may remove the on-body injector from the base 170 and attach the on-body injector 200 to the patient to administer the medicament.

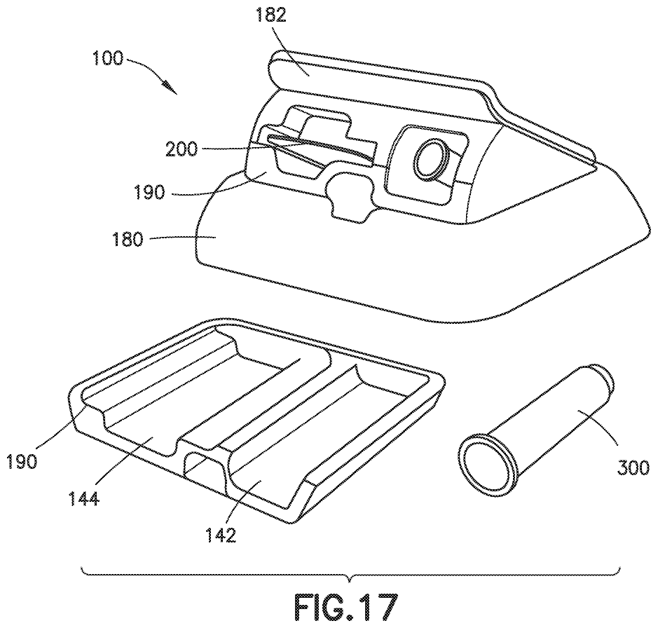

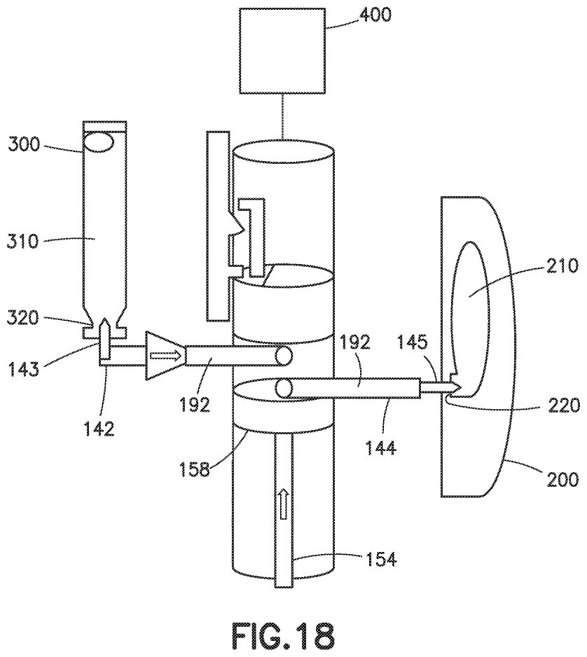

Referring now to FIGS. 17 and 18, an embodiment of the filling station 100 includes a base 180 and a lid 182 rotatable relative to the base. The lid 182 may be configured to receive a transfer tray 190 loaded with the on-body injector 200 and the fill cartridge 300. In particular, the lid 182 is configured to receive the tray 190 from the front side 183 of the lid 182 opposite the hinge 184. The user can depress the lid 182 into the base 180 once the tray 190 has been loaded to initiate the fill or refill operation. The tray 190 is functionally analogous to the tray 140 of the embodiment of FIG. 14, including an injector socket 144 for receiving and supporting the on-body injector 200, a cartridge socket 142 for receiving and supporting the fill cartridge 300, and a fluid path section 192 providing fluid communication between the cartridge socket 142 and the injector socket 144. As shown in FIG. 18, the cartridge socket 142 can include a needle 143 for piercing the septum or stopper 320 of the fill cartridge 300 to provide fluid communication between the fluid path section 192 and the interior chamber 310 of the fill cartridge 300. Similarly, the injector socket 144 can include a needle 145 for piercing the septum or port 220 of the on-body injector 200 to provide fluid communication between the fluid path section 192 and the medicament reservoir 210 of the on-body injector 200. The pump 154 in this embodiment includes a piston 158 to inject medicament from the fill cartridge 300 into the on-body injector 200 via the fluid path section 192.

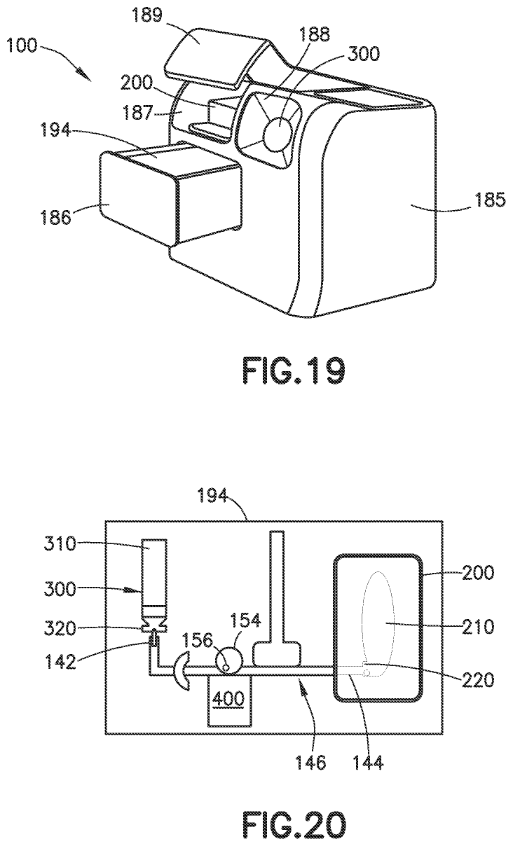

Referring now to FIGS. 19 and 20, an embodiment of the filling station 100 includes a base 185 having a drawer 186 for receiving the transfer tray 194, a first receptacle 187 for receiving the on-body injector 200, and a second receptacle 188 for receiving the fill cartridge 300. The first receptacle 187 may be shielded by a lid 189 to maintain sterility of the on body injector 200 during the fill or refill operation. As shown in FIG. 20, the transfer tray 194 is analogous to the transfer tray 140 of the embodiment of FIGS. 1-14. However, unlike the embodiment of FIGS. 1-14, the transfer tray 194 of the embodiment of FIGS. 19-20 is loaded into the drawer 186 prior to connecting the on-body injector 200 and the fill cartridge 300 to the transfer tray 194. With the transfer tray 194 loaded into the drawer 186, the transfer tray 194 aligns with the first receptacle 187 and the second receptacle 188 such that the on-body injector 200 and the fill cartridge 300 can be connected to the transfer tray 194 through the base 185. In particular, the on-body injector 200 can be inserted through the first receptacle 187 to connect to the injector socket 144 of the tray 194, and the fill cartridge 300 can be inserted through the second receptacle 188 to connect to the cartridge socket 142.

With continued reference to FIG. 20, the pump 154 may include a peristaltic pump similar to the embodiment of FIGS. 1-14. Performance of the fill or refill operation is also similar to that of the embodiment of FIGS. 1-14, with the controller 400 actuating the pump to inject medicament from the fill cartridge 300 into the on-body injector 200. The tray 194 and fill cartridge 300 may be disposed of after a predetermined number of uses (e.g. one use).

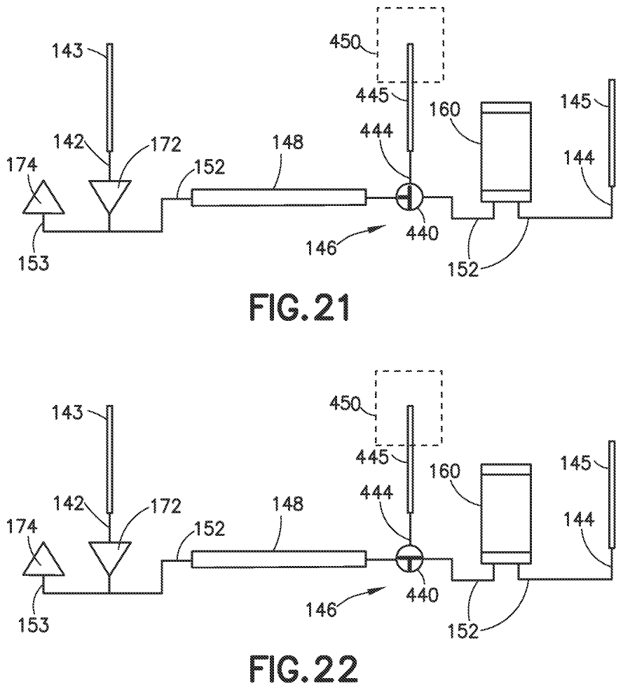

Referring now to FIGS. 21 and 22, some embodiments of the present disclosure include an auxiliary socket 444 configured for receiving an auxiliary cartridge 450. The auxiliary cartridge 450 may contain, in some examples, a drug that can be reconstituted, such as lyophilised powder. FIGS. 21 and 22 schematically show an embodiment of the transfer tray, identical to that of FIG. 3 except for the inclusion of the auxiliary cartridge 444 and associated components. The auxiliary socket 444 may include an auxiliary cartridge needle 445 configured to pierce a stopper or septum of the auxiliary cartridge 450 to establish fluid communication between the auxiliary cartridge 450 and the fluid path section 146. The fluid path section 146 includes at least one valve 440 for selectively providing fluid communication between the auxiliary socket 444 and the fluid path section 146. The valve 440 is located on an opposite side of the resilient tubing section 148 (which interfaces with the pump 154 as shown in FIG. 2) relative to the cartridge socket 142. The valve 440 may be moved to a first position, as shown in FIG. 21, to provide fluid communication between the cartridge socket 142 and the auxiliary socket 144, while isolating the injector socket 144. In this position, the pump 154 (see, e.g., FIG. 2) can be actuated to pump fluid from the fill cartridge 300 attached to the cartridge socket 142 into the auxiliary cartridge 450 attached to the auxiliary socket 444. The fluid pumped into the auxiliary cartridge 450 from the fill cartridge 300 may reconstitute the drug contained in the auxiliary cartridge 450. The resulting reconstituted drug may be pumped (via the pump 154) back and forth between the auxiliary cartridge 450 and the fill cartridge 300 to ensure full reconstitution of the drug. The fully reconstituted drug is then pumped a final time into the fill cartridge 300 for filling into the on-body injector 200.

Once the fully reconstituted drug is pumped to the fill cartridge 300, the valve 440 may be moved to a second position, as shown in FIG. 22, in which the cartridge socket 142 is in fluid communication with the injector socket 144. Also in the second position, the auxiliary socket 444 is isolated so that the reconstituted drug cannot reenter the auxiliary cartridge 450. In this position of the valve 440, the reconstituted drug may be pumped (via the pump 154) from the fill cartridge 300 to the on-body injector 200 in the same manner as described in connection with FIGS. 1-9. The at least one valve 440 may be moved between the first position of FIG. 21 and the second position of FIG. 22 automatically by the controller 400 (see FIG. 2) at a predetermined sequence.

The embodiments of the filling station 100 described herein may be particularly suited for filling and/or refilling the on-body injector 200. Examples of the on-body injector 200 are described in detail in U.S. Patent Application Publication No. 2020/0155755, the disclosure of which is hereby incorporated by reference in its entirety. However, embodiments of the filling station 100 may also be adapted for use with a variety of injector types, including those not limited to being worn on the body of the patient. As such, the embodiments of the filling station 100 described herein are not limited specifically to use in filling on-body injectors, or limited to any specific type, brand, or model of injector.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A system for filling an on-body injector, the system comprising:
the on-body injector having a medicament reservoir;
a fill cartridge having an internal chamber containing a medicament;
a filling station for receiving the on-body injector and the fill cartridge, the filling station comprising:
an injector socket for receiving the on-body injector;
a cartridge socket for receiving the fill cartridge;
a fluid path section providing fluid communication between the injector socket and the cartridge socket; and
a pump operatively engaging the fluid path section to transfer the medicament from the internal chamber of the fill cartridge to the medicament reservoir of the on-body injector.

2. The system of claim 1, wherein the pump comprises one or more rollers configured to engage a resilient tubing section of the fluid path section.

3. The system of claim 1, wherein the injector socket comprises a needle for piercing a septum or port of the on-body injector.

4. The system of claim 1, wherein the cartridge socket comprises a needle for piercing a septum or stopper of the fill cartridge.

5. The system of claim 1, wherein the injector socket, the cartridge socket, and the fluid path section are provided on a removable transfer tray.

6. The system of claim 5, wherein the filling station comprises:
a base for receiving the transfer tray; and
a lid pivotally connected to the base.

7. The system of claim 1, wherein the filling station comprises an inflation chamber configured to deliver a pre-fill inflation volume of air to the on-body injector.

8. The system of claim 7, wherein the pre-fill inflation volume of air is approximately 100 μL.

9. The system of claim 1, wherein the medicament reservoir of the on-body injector has a volume of approximately 3 mL.

10. The system of claim 1, wherein the filling station comprises a controller for actuating the pump.

11. The system of claim 10, wherein the controller is configured to actuate the pump to fill the medicament reservoir of the on-body injector to approximately 10 psi.

12. The system of claim 10, wherein the controller is configured to run the pump in reverse to evacuate air from the fluid path section.

13. The system of claim 12, wherein the filling station further comprises a reverse check valve through which the air is evacuated.

14. The system of claim 1, wherein the filling station further comprises:
an auxiliary socket configured for receiving an auxiliary cartridge; and
at least one valve movable between a first position and a second position,
wherein, in the first position of the at least one valve, the cartridge socket is in fluid communication with the auxiliary socket, and the injector socket is isolated, and
wherein, in the second position of the valve, the cartridge socket is in fluid communication with the injector socket.

15. A filling station for filling an on-body injector, the filling station comprising:
a removable transfer tray comprising:
an injector socket for receiving the on-body injector;
a cartridge socket for receiving a fill cartridge;
a fluid path section providing fluid communication between the injector socket and the cartridge socket; and
a pump operatively engaging the fluid path section to transfer the medicament from an internal chamber of the fill cartridge to a medicament reservoir of the on-body injector.

16. The filling station of claim 15, wherein the pump comprises one or more rollers configured to engage a resilient tubing section of the fluid path section.

17. The filling station of claim 15, wherein the injector socket comprises a needle for piercing a septum or port of the on-body injector.

18. The filling station of claim 15, wherein the cartridge socket comprises a needle for piercing a septum or stopper of the fill cartridge.

19. The filling station of claim 15, further comprising:
a base for receiving the transfer tray; and
a lid pivotally connected to the base.

20. The filling station of claim 15, further comprising an inflation chamber configured to deliver a pre-fill inflation volume of air to the on-body injector.

21. The filling station of claim 20, wherein the pre-fill inflation volume of air is approximately 100 μL.

22. The filling station of claim 15, wherein the filling station comprises a controller for actuating the pump, wherein the controller is configured to actuate the pump to fill the medicament reservoir of the on-body injector to approximately 10 psi.

23. The filling station of claim 22, wherein the controller is configured to run the pump in reverse to evacuate air from the fluid path section.
